(19) United States
(12) Patent Application Publication
(10) Pub. No.: US 2022/0289661 A1
(43) Pub. Date: Sep. 15, 2022

(54) CARBONYLATION-DEHYDRATION DUAL-FUNCTIONAL CATALYST PRECURSOR, PREPARATION METHOD THEEOF, CARBONYLATION-DEHYDRATION DUAL-FUNCTIONAL CATALYST AND USE THEREOF

(71) Applicant: Xiamen University, Xiamen (CN)

(72) Inventors: Nuowei Zhang, Xiamen (CN); Xinliang Lin, Xiamen (CN); Binghui Chen, Xiamen (CN); Lili Guo, Xiamen (CN); Fan Cai, Xiamen (CN); Songshou Ye, Xiamen (CN); Jianrong Xie, Xiamen (CN); Jinbao Zheng, Xiamen (CN)

(21) Appl. No.: 17/689,425

(22) Filed: Mar. 8, 2022

(30) Foreign Application Priority Data
Mar. 10, 2021 (CN) 202110259253.0

(51) Int. Cl. C07C 67/37; B01J 29/24; B01J 37/00; B01J 37/18

(52) U.S. Cl. CPC C07C 67/37; B01J 29/24; B01J 37/0009; B01J 37/18; B01J 2229/18

(57) ABSTRACT

A carbonylation-dehydration dual-functional catalyst precursor, a preparation method thereof, a carbonylation-dehydration dual-functional catalyst and use thereof are provided. The carbonylation-dehydration dual-functional catalyst precursor includes a modified silica-aluminum molecular sieve having an 8-member ring channel structure; a modified metal oxie loaded on the modified silica-aluminum molecular sieve having an 8-member ring channel structure by coupling, the coupling being performed using a silane coupling agent, wherein a modified component in the modified silica-aluminum molecular sieve having an 8-member ring channel structure includes at least one selected from the group consisting of copper oxide, zing oxide and iron oxide, and has a loading amount of 0.5-5 wt %, based on a metal mass of the modified component; and the modified metal oxie is prepared by modifying a composite metal oxide with an acid solution or an alkali solution, wherein the composite metal oxide is prepared based on a co-precipitation-calcination method.

### TECHNICAL FIELD

The present disclosure relates to the technical field of catalysts, and in particular to a carbonylation-dehydration dual-functional catalyst precursor, a preparation method thereof, a carbonylation-dehydration dual-functional catalyst and use thereof.

### BACKGROUND ART

In recent years, the process for producing ethanol from coal through synthesis gas has attracted wide attention and research. The process for producing ethanol from synthesis gas through dimethyl ether mainly includes four reaction steps: subjecting synthesis gas to a hydrogenation to obtain methanol, subjecting methanol to a dehydration to obtain dimethyl ether, subjecting dimethyl ether to a carbonylation in the presence of a molecular sieve to obtain methyl acetate, and subjecting methyl acetate to a hydrogenation to obtain ethanol and methanol. The core step in the process is subjecting dimethyl ether to a carbonylation to obtain methyl acetate, which is also the rate-limiting step of the entire reaction. Among them, the process for preparing dimethyl ether from methanol may result in the generation of water, which will inhibit the activity of the carbonylation of dimethyl ether and further reduce the reaction rate of the rate-limiting step. Moreover, in the prior art, when synthesis gas is used to prepare ethanol through dimethyl ether, molecular sieves with carbonylation function (such as MOR molecular sieves) are usually directly used as catalysts. The molecular sieves generally have dehydration ability, and thus under the conditions that the raw materials are unreasonable and the reaction conditions are unsuitable, the molecular sieve used in the carbonylation even preferentially undergoes a dehydration, which further limits the activity of carbonylation.

In the prior art, in order to avoid the influence of water generated during the process of dehydrating methanol to dimethyl ether on the carbonylation, dimethyl ether is usually directly used as the raw material to prepare methyl acetate, which does not fundamentally solve the problem existing in the production of ethanol from synthesis gas through dimethyl ether. In addition, in the prior art, in the production of ethanol from synthesis gas through dimethyl ether, increasing the temperature could resuce the influence of water in the system, but would shorten the service life of the molecular sieve, and meanwhile would lead to an increase in energy consumption.

### SUMMARY

An object of the present disclosure is to provide a carbonylation-dehydration dual-functional catalyst precursor, a preparation method thereof, a carbonylation-dehydration dual-functional catalyst and use thereof. When the carbonylation-dehydration dual-functional catalyst precursor provided by the present disclosure is activated in an H₂—N₂ mixed gas and used in the process for preparing methyl acetate from synthesis gas through dimethyl ether, it is possible to remove the water existing in the system and the water generated during the process of dehydrating methanol to dimethyl ether in time, thus realizing efficient conversion of dimethyl ether to methyl acetate; also, there is no need to increase the temperature of the system, and thus there is no problem of high energy consumption and short service life of molecular sieves.

In view of the above, the present disclosure provides the following technical solutions:

The present disclosure provides a carbonylation-dehydration dual-functional catalyst precursor, comprising

a modified silica-aluminum molecular sieve having an 8-member ring channel structure; and

a modified metal oxide loaded on the modified silica-aluminum molecular sieve having an 8-member ring channel structure by coupling, the coupling being performed using a silane coupling agent, wherein

a modified component in the modified silica-aluminum molecular sieve having an 8-member ring channel structure includes at least one selected from the group consisting of copper oxide, zinc oxide and iron oxide, and has a loading amount of 0.5-5 wt %, based on a metal mass of the modified component; and

the modified metal oxide is prepared by modifying a composite metal oxide with an acid solution or an alkali solution, wherein the composite metal oxide is prepared based on a co-precipitation-calcination method, and the composite metal oxide is at least one selected from the group consisting of CuₐZn₁₋ₐO_{y}, Cu_{b}Mn_{1-b}O_{y} and CuₘZnₙAl₁₋ₘ₋ₙO_{y}, wherein 0<a<1, 0<b<1, 0<m<1, 0<n<1, 0<m+n<1, and y=1.0-2.0.

In some embodiments, a molecular sieve in the modified silica-aluminum molecular sieve having an 8-member ring channel structure includes a mordenite (MOR) molecular sieve or a ferrierite (FER) molecular sieve.

In some embodiments, a mass ratio of the modified silica-aluminum molecular sieve having an 8-member ring channel structure to the modified metal oxide is in a range of 1:(0.05-0.5).

In some embodiments, the silane coupling agent includes at least one selected from the group consisting of 3-aminopropyltrimethoxysilane, 3-aminopropyltriethoxysilane, vinyltrichlorosilane, vinyl tris (2-methoxyethoxy) silane, and vinyltriacetoxysilane.

In some embodiments, the acid solution includes at least one selected from the group consisting of a hydrochloric acid solution, a nitric acid solution, a sulfuric acid solution, an acetic acid solution, and a citric acid solution, and has a concentration of 0.1-6 wt %.

In some embodiments, the alkali solution includes at least one selected from the group consisting of a sodium hydroxide solution, a potassium hydroxide solution, a calcium hydroxide solution and an ammonia water, and has a concentration of 0.1-8 wt %.

The present disclosure provides a method for preparing the carbonylation-dehydration dual-functional catalyst precursor described in the above technical solutions, comprising the following steps:

pretreating the modified metal oxide with the silane coupling agent to obtain a pretreated modified metal oxide; and

coupling the pretreated modified metal oxide and the modified silica-aluminum molecular sieve having an 8-member ring channel structure to obtain the carbonylation-dehydration dual-functional catalyst precursor.

In some embodiments, the silane coupling agent is used in a form of a silane coupling agent solution, and the silane coupling agent solution has a concentration of 0.5-50 wt %; a solvent of the silane coupling agent solution is an alcohol-water mixed solvent, and a volume ratio of the alcohol to water in the alcohol-water mixed solvent is in a range of (1-200):10.

In some embodiments, a ratio of the volume of the silane coupling agent solution to the total mass of the modified metal oxide and the modified silica-aluminum molecular sieve having an 8-member ring channel structure is in a range of (1-20) mL: 1 g.

The present disclosure provides a carbonylation-dehydration dual-functional catalyst, which is prepared by activating the carbonylation-dehydration dual-functional catalyst precursor described in the above technical solutions or the carbonylation-dehydration dual-functional catalyst precursor prepared by the method described in the above technical solutions in an H₂—N₂ mixed gas.

The present disclosure provides use of the carbonylation-dehydration dual-functional catalyst described in the above technical solutions in the production of methyl acetate from synthesis gas through dimethyl ether, wherein a reaction for producing methyl acetate through dimethyl ether is performed at a temperature of 170-280° C.

The present disclosure provides a carbonylation-dehydration dual-functional catalyst precursor, comprising a modified silica-aluminum molecular sieve having an 8-member ring channel structure, and a modified metal oxide loaded on the modified silica-aluminum molecular sieve having an 8-member ring channel structure by coupling, the coupling being performed by using a silane coupling agent, wherein a modified component in the modified silica-aluminum molecular sieve having an 8-member ring channel structure includes at least one selected from the group consisting of copper oxide, zinc oxide and iron oxide, and has a loading amount of 0.5-5 wt % based on a metal mass of the modified component; the modified metal oxide is prepared by modifying a composite metal oxide with an acid solution or an alkali solution, wherein the composite metal oxide is prepared based on a co-precipitation-calcination method, and the composite metal oxide is at least one selected from the group consisting of CuₐZn₁₋ₐO_{y}, Cu_{b}Mn_{1-b}O_{y} and CuₘZnₙAl₁₋ₘ₋ₙO_{y}, wherein 0<a<1, 0<b<1, 0<m<1, 0<n<1, 0<m+n<1, and y=1.0-2.0.

The carbonylation-dehydration dual-functional catalyst precursor according to the present disclosure is activated in an H₂—N₂ mixed gas to obtain a carbonylation-dehydration dual-functional catalyst, which has dual functions of carbonylation and dehydration, and in the production of methyl acetate from synthesis gas through dimethyl ether using the carbonylation-dehydration dual-functional catalyst, it is possible to remove the water existing in the system and the water produced during the process of dehydrating methanol to produce dimethyl ether in time, thus realizing efficient conversion of dimethyl ether to methyl acetate. Specifically, the modified silica-aluminum molecular sieve having an 8-member ring channel structure according to the present disclosure has excellent carbonylation ability for dimethyl ether, wherein after the modified silica-aluminum molecular sieve having an 8-member ring channel structure is modified by the modified component and then activated in an H₂—N₂ mixed gas, the modified components in the modified silica-aluminum molecular sieve having an 8-member ring channel structure are reduced to a low-valent state or zero-valent state, resulting in an active component (such as zero-valent copper) that could adsorb CO, which could promote the activation of CO, accelerate the generation of reaction intermediates, and accelerate the carbonylation reaction; the modified metal oxide has the ability of catalyzing the water-gas shift reaction, wherein after the composite metal oxide prepared by the co-precipitation-calcination method is modified by an acid solution or an alkali solution, and then activated in an H₂—N₂ mixed gas (wherein the copper in the modified metal oxide is reduced to zero-valent copper), it is possible to reduce the hydrogenation activity of the modified metal oxide, and is beneficial to avoiding the formation of by-products (alkanes) from the hydrogenation reaction. Moreover, in the present disclosure, the modified silica-aluminum molecular sieve having an 8-member ring channel structure has a reaction temperature similar to that of the modified metal oxide, and uses a modified silica-aluminum molecular sieve having an 8-member ring channel structure with dimethyl ether carbonylation ability as a main body, and the main body is coupled with a modified metal oxide having water-gas shift reaction capability by a silane coupling agent to form a carbonylation-dehydration dual-functional catalyst precursor, and then the precursor is activated in an H₂—N₂ mixed gas to obtain a carbonylation-dehydration dual-functional catalyst, which could give full play to the synergy between different active components, eliminate the adverse effect of the H₂O in the system and the generated by-product H₂O on the carbonylation reaction, and ensure the carbonylation activity of the modified silica-aluminum molecular sieve having an 8-member ring channel structure, thereby realizing efficient conversion of dimethyl ether to methyl acetate, and improving the production efficiency of the environmental kindly route. Further, the catalyst has extremely high industrial value and could be directly applied to coal gasification equipment. Compared with the process of directly preparing methyl acetate from dimethyl ether in the prior art, the catalyst has a significantly improved industrial value, and avoids the problems (such as high energy consumption and short service life of molecular sieve) caused by increasing the temperature to reduce the adverse effects of water in the system in the prior art, and is more suitable for large-scale production.

The present disclosure provides a method for preparing the carbonylation-dehydration dual-functional catalyst precursor, which is simple in operation and could realize large-scale preparation.

In addition, when the carbonylation-dehydration dual-functional catalyst according to the present disclosure is used in actual industrial production, the existing industrial equipment could be used to directly replace the original molecular sieve catalyst with the carbonylation-dehydration dual-functional catalyst, without additional equipment, which improves the overall carbonylation capacity while reducing equipment investment and operating costs, thus having extremely high industrial value and practical significance.

### DETAILED DESCRIPTION OF THE EMBODIMENTS

The present disclosure provides a carbonylation-dehydration dual-functional catalyst precursor, comprising

a modified silica-aluminum molecular sieve having an 8-member ring channel structure; and

a modified metal oxide loaded on the modified silica-aluminum molecular sieve having an 8-member ring channel structure by coupling, the coupling being performed by using a silane coupling agent, wherein

a modified component in the modified silica-aluminum molecular sieve having an 8-member ring channel structure includes at least one selected from the group consisting of copper oxide, zinc oxide and iron oxide, and has a loading amount of 0.5-5 wt %, based on metal mass in the modified component; and

the modified metal oxide is prepared by modifying a composite metal oxide with an acid solution or an alkali solution, wherein the composite metal oxide is prepared based on a co-precipitation-calcination method, and the composite metal oxide is at least one selected from the group consisting of CuₐZn₁₋ₐO_{y}, Cu_{b}Mn_{1-b}O_{y} and CuₘZnₙAl₁₋ₘ₋ₙO_{y}, wherein 0<a<1, 0<b<1, 0<m<1, 0<n<1, 0<m+n<1, and y=1.0-2.0.

The carbonylation-dehydration dual-functional catalyst precursor according to the present disclosure comprises a modified silica-aluminum molecular sieve having an 8-member ring channel structure, wherein a modified component in the modified silica-aluminum molecular sieve having an 8-member ring channel structure includes at least one selected from the group consisting of copper oxide, zinc oxide and iron oxide, preferably copper oxide, zinc oxide and iron oxide, and more preferably copper oxide; the modified component in the modified silica-aluminum molecular sieve having an 8-member ring channel structure has a loading amount of 0.5-5 wt %, preferably 1-4 wt %, and more preferably 2-3 wt %, based on a metal mass of the modified component. In some embodiments of the present disclosure, a molecular sieve in the modified silica-aluminum molecular sieve having an 8-member ring channel structure includes at least one selected from the group consisting of an MOR molecular sieve and an FER molecular sieve, and preferably an MOR molecular sieve. In an embodiment of the present disclosure, a copper oxide modified MOR molecular sieve is specifically described as an example, and the loading amount of Cu in the copper oxide modified MOR molecular sieve is 2 wt %.

In some embodiments of the present disclosure, the method for preparing the modified silica-aluminum molecular sieve having an 8-member ring channel structure includes the following steps:

subjecting a molecular sieve to an ammonium ion exchange treatment, and subjecting the obtained ammonia ion exchange molecular sieve to a first calcination to obtain an H-type molecular sieve; and

subjecting the H-type molecular sieve to a metal ion exchange treatment, and subjecting the obtained metal ion exchange molecular sieve to a second calcination to obtain the modified silica-aluminum molecular sieve having an 8-member ring channel structure.

In the present disclosure, there is no special limitation on the source of the molecular sieve, and commercially available products well known to those skilled in the art may be used.

In some embodiments of the present disclosure, a reagent required for the ammonium ion exchange treatment is ammonium chloride or ammonium nitrate; the reagent required for the ammonium ion exchange treatment is used in a form of an ammonium salt aqueous solution, and the ammonium salt aqueous solution has a concentration of 0.05-0.5 mol/L, and preferably 0.1-0.2 mol/L; a ratio of the ammonium salt aqueous solution to the molecular sieve is in a range of (35-60) mL: 1 g, and preferably (40-50) mL: 1 g. In some embodiments of the present disclosure, the ammonium ion exchange treatment is performed at a temperature of 40-100° C., and preferably 60-80° C.; the ammonium ion exchange treatment is performed for 2-6 h, and preferably 3-4 h. In some embodiments of the present disclosure, after the ammonium ion exchange treatment, the method further includes: filtering the product system obtained after the ammonium ion exchange treatment, and washing the obtained filter cake with deionized water to obtain an ammonia ion exchange molecular sieve. In some embodiments of the present disclosure, the first calcination is performed at a temperature of 450-600° C., and preferably 500-550° C.; the first calcination is performed for 3-6 h, and preferably 4-5 h; and the first calcination is performed in an air atmosphere.

In some embodiments of the present disclosure, a reagent required for the metal ion exchange treatment is a hydrated metal nitrate compound, and specifically may be one selected from the group consisting of copper nitrate trihydrate, copper nitrate hexahydrate, zinc nitrate hexahydrate and iron nitrate nonahydrate; the hydrated metal nitrate compound is used in a form of an aqueous nitrate solution. In some embodiments, the aqueous nitrate solution has a concentration of 0.02-0.3 mol/L, and preferably 0.05-0.1 mol/L; a ratio of the aqueous nitrate solution to the molecular sieve is in a range of (40-60) mL:1 g, and preferably (45-50) mL:1 g. In some embodiments of the present disclosure, the metal ion exchange treatment is performed at a temperature of 40-100° C., and preferably 60-80° C.; the metal ion exchange treatment is performed for 4-8 h, and preferably 4-6 h. In some embodiments of the present disclosure, after the metal ion exchange treatment, the method further includes: filtering the product system obtained after the metal ion exchange treatment, and washing the obtained filter cake with deionized water to obtain a metal ion exchange molecular sieve. In some embodiments of the present disclosure, the second calcination is performed at a temperature of 450-650° C., and preferably 500-550° C.; the second calcination is performed for 4-8 h, and preferably 5-6 h; and the second calcination is performed in an air atmosphere.

In the present disclosure, the modified silica-aluminum molecular sieve having an 8-member ring channel structure has dimethyl ether carbonylation ability and could catalyze the carbonylation of dimethyl ether to obtain methyl acetate.

The carbonylation-dehydration dual-functional catalyst precursor according to the present disclosure comprises a modified metal oxide loaded on the modified silica-aluminum molecular sieve having an 8-member ring channel structure by coupling, and the coupling is performed by using a silane coupling agent. In the present disclosure, the modified metal oxide is prepared by modifying a composite metal oxide with an acid solution or an alkali solution; the composite metal oxide is prepared based on a co-precipitation-calcination method, and the composite metal oxide is at least one selected from the group consisting of CuₐZn₁₋ₐO_{y}, Cu_{b}Mn_{1-b}O_{y} and CuₘZnₙAl₁₋ₘ₋ₙO_{y}, wherein 0<a<1, 0<b<1, 0<m<1, 0<n<1, 0<m+n<1, and y=1.0-2.0. In some embodiments of the present disclosure, the composite metal oxide is at least one selected from the group consisting of a copper-zinc composite oxide, a copper-manganese composite oxide, and a copper-zinc-aluminum composite oxide; a molar ratio of copper element to zinc element in the copper-zinc composite oxide is in a range of 1:(0.5-1.5), and preferably 1:1, a molar ratio of copper element to manganese element in the copper-manganese composite oxide is in a range of 1:(0.2-1.5), and preferably 1:1, and a molar ratio of copper element to zinc element to aluminum element in the copper-zinc-aluminum composite oxide is in a range of 1:(0.3-1.5): (0.05-0.5), and preferably 1:(0.4-0.8):(0.1-0.3).

In some embodiments of the present disclosure, the composite metal oxide is prepared by a method including the following steps:

adding an aqueous solution of soluble metal salts and an aqueous solution of a precipitant into water, and subjecting the resulting mixture to a co-precipitation to obtain a precipitation material; and

subjecting the precipitation material to a third calcination to obtain the composite metal oxide.

In some embodiments of the present disclosure, the aqueous solution of the soluble metal salts and the aqueous solution of the precipitant are added into water, and subjected to the co-precipitation to obtain the precipitation material. In some embodiments of the present disclosure, the soluble metal salts are nitrates, and the specific type of the soluble metal salts corresponds to the type of the metals in the composite metal oxide. Specifically, for example, under the condition that the composite metal oxide is a copper-zinc composite oxide, the soluble metal salts are copper nitrate and zinc nitrate; under the condition that the composite metal oxide is a copper-manganese composite oxide, the soluble metal salts are copper nitrate and manganese nitrate; under the condition that the metal oxide is a copper-zinc-aluminum composite oxide, the soluble metal salts are copper nitrate, zinc nitrate, and aluminum nitrate. In the present disclosure, a specific ratio of multiple nitrates in the soluble metal salts is controlled to ensure that each metal element in the composite metal oxide satisfies the above ratio. In some embodiments of the present disclosure, the aqueous solution of the soluble metal salts has a concentration of 1-3 mol/L, and preferably 1.5-2.5 mol/L, wherein the concentration of the aqueous solution of the soluble metal salts is specifically a total molar concentration of the soluble metal salts.

In some embodiments of the present disclosure, the precipitant includes at least one selected from the group consisting of sodium carbonate, sodium bicarbonate and potassium carbonate, and preferably sodium carbonate. In some embodiments of the present disclosure, the aqueous solution of the precipitant has a concentration of 6-13 wt %, and preferably 8-11 wt %.

In some embodiments of the present disclosure, a volume ratio of water to the aqueous solution of the soluble metal salts to the aqueous solution of the precipitant is in a range of 1: (0.5-1.5):(1.5-2.5), and preferably 1:(0.8-1.2):(1.8-2.2). In some embodiments of the present disclosure, the aqueous solution of the soluble metal salts and the aqueous solution of the precipitant are dropped into water, wherein the aqueous solution of the soluble metal salts and the aqueous solution of the precipitant are dropped at such a rate that ensures a pH of the system to be 6-8, and preferably 7. In some embodiments of the present disclosure, during the dropping process, the system is kept at a temperature of 50-100° C., and preferably 60-80° C. In some embodiments of the present disclosure, after the dropping is completed, the obtained system is stirred for 2-4 h with heat preservation to achieve sufficient co-precipitation. In some embodiments of the present disclosure, after the co-precipitation is completed, the obtained system is filtered, and the obtained filter cake is washed with deionized water, and the washed filter cake is dried to obtain a precipitation material. In some embodiments of the present disclosure, the washed filter cake is dried at a temperature of 100-120° C., and preferably 110° C.; and the washed filter cake is dried for 20-30 h, and preferably 24 h.

After the precipitation material is obtained, the precipitation material is subjected to a third calcination to obtain a composite metal oxide. In some embodiments of the present disclosure, the third calcination is performed at a temperature of 250-600° C., and preferably 350-450° C.; the third calcination is performed for 1-6 h, and preferably 4-5 h; and the third calcination is preferably performed in air.

In the present disclosure, the composite metal oxide is prepared based on a co-precipitation-calcination method, which is convenient for adjusting the types and proportions of metal elements in the composite metal oxide. Besides, based on the incorporation of different metals, it is possible to change the distribution of different metals on the surface of the composite metal oxide and give full play to the synergy between metals to ensure a better reactivity.

In the present disclosure, the modified metal oxide is prepared by modifying a composite metal oxide with an acid solution or an alkali solution; the acid solution includes at least one selected from the group consisting of hydrochloric acid, a nitric acid solution, an acetic acid solution and a citric acid solution, and preferably hydrochloric acid and a nitric acid solution; the acid solution has a concentration of 0.1-6 wt %, and preferably 4-5 wt %. In some embodiments of the present disclosure, the alkali solution includes at least one selected from the group consisting of a sodium hydroxide solution, a potassium hydroxide solution, a calcium hydroxide solution and an ammonia water, and the alkali solution has a concentration of 0.1-8 wt %, and preferably 4-5 wt %.

In some embodiments of the present disclosure, the modified metal oxide is prepared by a method including the following steps:

placing the composite metal oxide in an acid solution or an alkali solution, and subjecting the resulting mixture to a modification treatment to obtain the modified metal oxide.

In the present disclosure, there is no special limitation on the ratio of the composite metal oxide to the acid solution or alkali solution, and the acid solution or alkali solution which could completely immerse the composite metal oxide may be used. In some embodiments of the present disclosure, the modification treatment is performed at a temperature of 20-40° C., and preferably 25-35° C.; in the Examples of the present disclosure, the modification treatment is carried out at ambient temperature, i.e. no additional heating or cooling is required. In some embodiments of the present disclosure, the modification treatment is performed for 2-24 h, and preferably 2-5 h.

In the present disclosure, the original structure of the composite metal oxide could be partially destroyed by using an acid solution or an alkali solution to modify the composite metal oxide, thereby achieving the purpose of limiting the hydrogenation ability of the composite metal oxide and avoiding the problem that the distribution of the final product will be adversely affected by hydrogenation reaction when the carbonylation-dehydration dual-functional catalyst precursor is activated in an H₂—N₂ mixed gas and then used in the producing of methyl acetate from synthesis gas through dimethyl ether.

In some embodiments, in the carbonylation-dehydration dual-functional catalyst precursor according to the present disclosure, a mass ratio of the modified silica-aluminum molecular sieve having an 8-member ring channel structure to the modified metal oxide is in a range of 1:(0.05-0.5), and preferably 1:(0.05-0.2). In the present disclosure, the modified silica-aluminum molecular sieve having an 8-member ring channel structure and the modified metal oxide are coupled by a silane coupling agent. In some embodiments, the silane coupling agent includes at least one selected from the group consisting of 3-aminopropyltrimethoxysilane, 3-aminopropyltriethoxysilane, vinyltrichlorosilane, vinyl tris (2-methoxyethoxy) silane and vinyltriacetoxysilane, preferably 3-aminopropyltrimethoxysilane, 3-aminopropyltriethoxysilane and vinyltrichlorosilane, and more preferably 3-aminopropyltrimethoxysilane.

In the present disclosure, the modified silica-aluminum molecular sieve having an 8-member ring channel structure and the modified metal oxide are coupled by a silane coupling agent to form a carbonylation-dehydration dual-functional catalyst precursor, and the carbonylation-dehydration dual-functional catalyst precursor is activated in an H₂—N₂ mixed gas to obtain a carbonylation-dehydration dual-functional catalyst. The catalyst could give full play to the synergy between the different active components, eliminate the adverse effects of the H₂O in the system and the generated by-product H₂O on the carbonylation reaction, and ensure the carbonylation activity of the modified silica-aluminum molecular sieve having an 8-member ring channel structure, thereby realizing the efficient conversion of dimethyl ether to methyl acetate.

The present disclosure provides a method for preparing the carbonylation-dehydration dual-functional catalyst precursor described in the above technical solutions, comprising the following steps:

pretreating the modified metal oxide with the silane coupling agent to obtain a pretreated modified metal oxide; and

coupling the pretreated modified metal oxide and the modified silica-aluminum molecular sieve having an 8-member ring channel structure to obtain the carbonylation-dehydration dual-functional catalyst precursor.

In the present disclosure, a silane coupling agent is used to pretreat the modified metal oxide to obtain a pretreated modified metal oxide. In some embodiments of the present disclosure, the silane coupling agent is used in a form of a silane coupling agent solution, and the silane coupling agent solution has a concentration of 0.5-50 wt %, and preferably 10-20 wt %; a solvent of the silane coupling agent solution is an alcohol-water mixed solvent, and a volume ratio of the alcohol to water in the alcohol-water mixed solvent is in a range of (1-200):10, preferably (4-100):10, and more preferably (6-30):10. In some embodiments of the present disclosure, the alcohol in the alcohol-water mixed solvent is a small-molecule alcohol solvent, and preferably at least one selected from the group consisting of methanol, ethanol, propanol and isopropanol, and more preferably ethanol. In some embodiments of the present disclosure, a ratio of the volume of the silane coupling agent solution to the total mass of the modified metal oxide and the modified silica-aluminum molecular sieve having an 8-member ring channel structure is in a range of (1-20) mL:1 g, and preferably (1.5-5) mL:1 g.

In the present disclosure, the silane coupling agent solution is mixed with the modified metal oxide for a pretreatment. In some embodiments, the pretreatment is performed at a temperature of 20-80° C., and preferably 25-40° C.; in the Examples of the present disclosure, the pretreatment is performed at ambient temperature, i.e., no additional heating or cooling is required. In some embodiments of the present disclosure, the pretreatment is performed for 10 min-5 h, and preferably 0.5-2 h. In some embodiments of the present disclosure, the pretreatment is performed under stirring condition.

In some embodiments of the present disclosure, the system obtained after the pretreatment may be filtered, the obtained filter cake is mixed with the modified silica-aluminum molecular sieve having an 8-member ring channel structure, and the resulting mixture is subjected to a coupling (denoted as a first coupling) to obtain the carbonylation-dehydration dual-functional catalyst precursor; also, in some embodiments, the system obtained after the pretreatment may be mixed with the modified silica-aluminum molecular sieve having an 8-member ring channel structure (denoted as a second coupling) to obtain a carbonylation-dehydration dual-functional catalyst precursor. The two cases are described below respectively.

In some embodiments of the present disclosure, the first coupling includes a grinding and a stirring performed in sequence; the first coupling is totally performed for 2-4 h. In the present disclosure, there is no special limitation on the time of grinding and stirring, as long as the components could be fully mixed to be uniform and the coupling could be completed. In some embodiments, the first coupling treatment is performed at a temperature of 30-60° C. In some embodiments of the present disclosure, after the first coupling, the obtained material is tableted and then sieved to obtain a carbonylation-dehydration dual-functional catalyst precursor with a particle size of 20-40 mesh.

In some embodiments of the present disclosure, the second coupling is performed at a temperature of 20-80° C., and preferably 25-40° C. In the Examples of the present disclosure, the pretreatment is performed at ambient temperature, i.e., no additional heating or cooling is required. In some embodiments of the present disclosure, the pretreatment is performed for 0.5-4 h, and preferably 1-2 h. In some embodiments of the present disclosure, the second coupling is performed under stirring condition. In some embodiments of the present disclosure, after the second coupling treatment, the obtained system is filtered, the obtained filter cake is dried, the obtained material after drying is tableted and then sieved to obtain a carbonylation-dehydration dual-functional catalyst precursor with a particle size of 20-40 mesh. In some embodiments of the present disclosure, the drying is performed at a temperature of 70-90° C., and preferably 80° C.; the drying is performed for 1-3 h, and preferably 2 h.

The present disclosure provides a carbonylation-dehydration dual-functional catalyst, which is obtained by activating the carbonylation-dehydration dual-functional catalyst precursor described in the above technical solutions or the carbonylation-dehydration dual-functional catalyst precursor prepared by the method described in the above technical solutions in an H₂—N₂ mixed gas. In some embodiments of the present disclosure, a volume fraction of H₂ in the H₂—N₂ mixed gas is in a range of 4-6%, and preferably 5%. In some embodiments of the present disclosure, the carbonylation-dehydration dual-functional catalyst precursor is activated at a temperature of 150-400° C., and preferably 200-260° C.; the carbonylation-dehydration dual-functional catalyst precursor is activated for 2-12 h, and preferably 5-8 h. In the present disclosure, the metal oxide in the carbonylation-dehydration dual-functional catalyst precursor is reduced to a low-valent state or zero-valent state through activation, and the finally obtained carbonylation-dehydration dual-functional catalyst has good dual functions of carbonylation and dehydration; in the carbonylation-dehydration dual-functional catalyst, copper and iron exist in a form of simple substances, and other metals still exist in a form of metal oxides.

The present disclosure provides use of the carbonylation-dehydration dual-functional catalyst of the above technical solutions in the production of methyl acetate from synthesis gas through dimethyl ether, wherein a reaction for producing methyl acetate through dimethyl ether is performed at a temperature of 170-280° C. In some embodiments of the present disclosure, the synthesis gas includes dimethyl ether, CO and H₂O, wherein a volume ratio of CO to dimethyl ether is in a range of (2-50):1, and preferably (5-20):1, more preferably (8-10):1, and a volume ratio of CO to H₂O is in a range of (5-100):1, and preferably (10-50):1, and more preferably (15-45):1.

In some embodiments of the present disclosure, a method for using the carbonylation-dehydration dual-functional catalyst includes the following steps:

in the presence of the carbonylation-dehydration dual-functional catalyst, using synthesis gas as a raw material to prepare methyl acetate.

In some embodiments of the present disclosure, using synthesis gas as a raw material to prepare methyl acetate is performed under the following reaction conditions: a temperature of 170-280° C., preferably 190-210° C.; a pressure of 1-5 MPa, preferably 2-3 MPa; and a space velocity of 1000-8000 mL/g/h, preferably 1000-3000 mL/g/h.

The technical solutions of the present disclosure will be clearly and completely described below in conjunction with the embodiments of the present disclosure. Obviously, the described embodiments are only a part of the embodiments of the present disclosure, rather than all the embodiments. Based on the embodiments of the present disclosure, all other embodiments obtained by those of ordinary skill in the art without creative labor shall fall within the protection scope of the present disclosure.

A method for preparing a copper oxide modified MOR molecular sieve used in the following examples and comparative examples is performed as follows:

10 g of MOR molecular sieve was mixed with 500 mL of an ammonium nitrate solution with a concentration of 0.2 mol/L, and the resulting mixture was subjected to an ammonium ion exchange treatment at a temperature of 80° C. for 4 h. The resulting product system was filtered, and the obtained filter cake was washed with deionized water, obtaining an ammonia ion exchange molecular sieve. The ammonia ion exchange molecular sieve was calcined in air at a temperature of 550° C. for 5 h, obtaining an H-type molecular sieve.

The H-type molecular sieve was mixed with 500 mL of copper nitrate trihydrate solution with a concentration of 0.05 mol/L, and the resulting mixture was subjected to a metal ion exchange treatment at a temperature of 80° C. for 4 h. The resulting product system was filtered, and the obtained filter cake was washed with deionized water, obtaining a metal ion exchange molecular sieve. The metal ion exchange molecular sieve was calcined in air at a temperature of 550° C. for 5 h, obtaining a copper oxide modified MOR molecular sieve.

### EXAMPLE 1

(1) 9.7 g of copper nitrate trihydrate and 11.9 g of zinc nitrate hexahydrate were weighed and dissolved in 50 mL of deionized water, obtaining a copper nitrate-zinc nitrate mixed solution. 8.7 g of anhydrous sodium carbonate was weighed and dissolved in 100 mL of deionized water, obtaining a sodium carbonate solution. 50 mL of deionized water was added into the round-bottom flask, the copper nitrate-zinc nitrate mixed solution and the sodium carbonate solution were added dropwise into the round-bottom flask at a temperature of 60° C. at a dropping rate that was enough to maintain the pH of the system at 7. After the completion of dropping, the reaction system was stirred for 3 h with a heat preservation. The system obtained after the reaction was filtered, the obtained filter cake was washed with deionized water and then placed in an oven at a temperature of 110° C. for 24 h. The dried solid was calcined in air at a temperature of 450° C. for 5 h, obtaining a composite metal oxide (specifically being a copper-zinc composite oxide, with a molar ratio of copper to zinc being 1:1).

The composite metal oxide was added into an HNO₃ solution with a concentration of 5 wt % and immersed for 2 h at ambient temperature (25° C.), obtaining a modified metal oxide.

(2) 3-aminopropyltriethoxysilane, deionized water and absolute ethanol were mixed at a mass ratio of 20:50:30, obtaining a silane coupling agent mixed solution.

(3) 0.5 g of the modified metal oxide was added into 20 mL of the silane coupling agent mixed solution, and stirred at ambient temperature for 1 h. 10 g of copper oxide modified MOR molecular sieve (with a Cu loading of 2.0 wt %) was then added thereto, and continuously stirred at ambient temperature for 1 h. The resulting system was then filtered, and the obtained filter cake was placed in an oven at a temperature of 80° C. for 2 h, then tableted and sieved sequentially, obtaining a dual-functional catalyst precursor with a particle size of 20-40 mesh, which was denoted as Cat1.

### EXAMPLE 2

(1) 14.5 g of copper nitrate trihydrate, 8.9 g of zinc nitrate hexahydrate and 3.8 g of aluminum nitrate nonahydrate were weighed and dissolved in 50 mL of deionized water, obtaining a copper nitrate-zinc nitrate-aluminum nitrate mixed solution. 11.5 g of anhydrous sodium carbonate was weighed and dissolved in 100 mL of deionized water, obtaining a sodium carbonate solution. 50 mL of deionized water was added into a round-bottom flask, the copper nitrate-zinc nitrate-aluminum nitrate mixed solution and the sodium carbonate solution were added dropwise into the round-bottom flask at a temperature of 60° C. at a dropping rate that was enough to maintain the pH of the system at 7. After the completion of dropping, the reaction system was stirred for 3 h with a heat preservation. The system obtained after the reaction was filtered, the obtained filter cake was washed with deionized water and then placed in an oven at a temperature of 110° C. for 24 h. The dried solid was calcined in air at a temperature of 450° C. for 5 h, obtaining a composite metal oxide (specifically being a copper-zinc-aluminum composite oxide, with a molar ratio of copper to zinc to aluminum being 6:3:1). The composite metal oxide was added into an HNO₃ solution with a concentration of 5 wt % and immersed for 2 h at ambient temperature (25° C.), obtaining a modified metal oxide.

(2) 3-aminopropyltriethoxysilane, deionized water and absolute ethanol were mixed at a mass ratio of 20:50:30, obtaining a silane coupling agent mixed solution.

(3) 0.5 g of the modified metal oxide was added into 20 mL of the silane coupling agent mixed solution, stirred at ambient temperature for 1 h, then 10 g of the copper oxide modified MOR molecular sieve was added thereto, and continuously stirred at ambient temperature for 1 h. The resulting system was then filtered, and the obtained filter cake was placed in an oven at a temperature of 80° C. for 2 h, then tableted and sieved sequentially, obtaining a dual-functional catalyst precursor with a particle size of 20-40 mesh, which was denoted as Cat2.

### EXAMPLE 3

(1) 9.7 g of copper nitrate trihydrate, and 10.1 g of manganese nitrate tetrahydrate were weighed and dissolved in 50 mL of deionized water, obtaining a copper nitrate-manganese nitrate mixed solution. 8.8 g of anhydrous sodium carbonate was weighed and dissolved in 100 mL of deionized water, obtaining a sodium carbonate solution. 50 mL of deionized water was added into a round-bottom flask, and the copper nitrate-manganese nitrate mixed solution and the sodium carbonate solution were added dropwise into the round-bottom flask at a temperature of 60° C. at a dropping rate that was enough to maintain the pH of the system at 7. After the completion of dropping, the reaction system was stirred for 3 h with a heat preservation. The system obtained after the reaction was filtered, the obtained filter cake was washed with deionized water and then placed in an oven at a temperature of 110° C. for 24 h. The dried solid was calcined in air at a temperature of 450° C. for 5 h, obtaining a composite metal oxide (specifically being a copper-manganese composite oxide, with a molar ratio of copper to manganese being 1:1); and the composite metal oxide was added into an HNO₃ solution with a concentration of 5 wt % and immersed for 2 h, obtaining a modified metal oxide.

(2) 3-aminopropyltriethoxysilane, deionized water and absolute ethanol were mixed at a mass ratio of 20:50:30, obtaining a silane coupling agent mixed solution.

(3) 0.5 g of the modified metal oxide was added into 20 mL of the silane coupling agent mixed solution, and stirred at ambient temperature for 1 h. 10 g of copper oxide modified MOR molecular sieve was added thereto, and continuously stirred at ambient temperature for 1 h. The resulting system was then filtered, and the obtained filter cake was placed in an oven at a temperature of 80° C. for 2 h, then tableted and sieved sequentially, obtaining a dual-functional catalyst precursor with a particle size of 20-40 mesh, which was denoted as Cat3.

### COMPARATIVE EXAMPLE 1

A modified metal oxide was prepared according to the method of Example 1. Then 0.5 g of the modified metal oxide and 10 g of the copper oxide modified MOR molecular sieve were mixed and ground for 1 h. The resulting mixture was tableted and then sieved, obtaining a catalyst precursor with the particle size of 20-40 mesh, which was denoted as Cat1#.

### COMPARATIVE EXAMPLE 2

A modified metal oxide was prepared according to the method of Example 2. Then 0.5 g of the modified metal oxide and 10 g of the copper oxide modified MOR molecular sieve were mixed and ground for 1 h. The resulting mixture was tableted and then sieved, obtaining a catalyst precursor with the particle size of 20-40 mesh, which was denoted as Cat2#.

### COMPARATIVE EXAMPLE 3

A modified metal oxide was prepared according to the method of Example 3. Then 0.5 g of the modified metal oxide and 10 g of the copper oxide modified MOR molecular sieve were mixed and ground for 1 h. The resulting mixture was tableted and then sieved, obtaining a catalyst precursor with the particle size of 20-40 mesh, which was denoted as Cat3#.

### COMPARATIVE EXAMPLE 4

10 g of the copper oxide modified MOR molecular sieve was tableted and ground, obtaining a catalyst precursor with the particle size of 20-40 mesh, which was denoted as Cat#.

### USE EXAMPLE 1

The dual-functional catalyst precursor prepared in Example 1 was activated in an H₂—N₂ mixed gas and then used in a reaction for producing methyl acetate from synthesis gas through dimethyl ether, and the reaction is performed as follows:

2.5 g of the dual-functional catalyst precursor was packed into a quartz tube. The quartz tube was then placed into a fixed-bed tubular reactor, and the H₂—N₂ mixed gas (with a volume fraction of H₂ being 5%, a volume fraction of N₂ being 95%) was introduced into the reactor. The dual-functional catalyst precursor was activated at a temperature of 260° C. for 5 h, and then a reaction raw material synthesis gas (specifically being a dimethyl ether-CO—H₂O mixed gas, with a volume fraction of dimethyl ether being 10%, a volume fraction of CO being 88%, and a volume fraction of H₂O being 2%) was introduced into the reactor, and subjected to a reaction at a temperature of 210° C., a pressure of 2 MPa, and a space velocity of 1000 mL/g/h.

The performance of the catalyst precursors prepared in Examples 2-3 and Comparative Examples 1-4 were tested according to the above method, and the test results are shown in Table 1.

TABLE 1
Test results of the performance of the catalyst precursors prepared in Examples 1-3 and Comparative Examples 1-4 after activation

| Catalyst | Mass selectivity % | | CO |
|---|---|---|---|
| precursor | Dimethyl ether | Methyl acetate | conversion % |
| Cat1 | 40.2 | 58.9 | 7.6 |
| Cat1^{#} | 97.8 | 1.1 | 0.4 |
| Cat2 | 10.3 | 88.7 | 11.4 |
| Cat2^{#} | 93.1 | 6.8 | 1.3 |
| Cat3 | 29.0 | 69.9 | 9.0 |
| Cat3^{#} | 95.4 | 3.9 | 1.2 |
| Cat^{#} | 46.6 | 52.1 | 6.0 |

It can be seen from Table 1 that compared with the catalyst prepared by mechanically mixing the modified metal oxide and metal modified MOR molecular sieve, the dual-functional catalyst prepared based on the silane coupling agent of the present disclosure has greatly improved CO conversion and methyl acetate selectivity, indicating that the dual-functional catalyst coupled with silane coupling agent could better play role of the synergistic effect between the active components and promote the progress of the reaction. Moreover, according to Table 1, it can be seen that compared with the catalyst prepared by only using copper oxide modified MOR molecular sieve, the dual-functional catalyst obtained by coupling the composite modified metal oxide (i.e. water gas shift catalyst) with a silane coupling agent has an improved catalytic effect, while the catalyst prepared by mechanically mixing the copper oxide modified MOR molecular sieve with the modified metal oxide basically loses the carbonylation activity, which is caused by the competition between the water gas shift reaction and the carbonylation reaction for CO.

### EXAMPLE 4

A dual-functional catalyst precursor was prepared according to the method of Example 2, except that 3-aminopropyltriethoxysilane was replaced with 3-aminopropyltrimethoxysilane. The final obtained dual-functional catalyst precursor was denoted as Cat4.

### EXAMPLE 5

A dual-functional catalyst precursor was prepared according to the method of Example 2, except that 3-aminopropyltriethoxysilane was replaced with vinyltrichlorosilane. The final obtained dual-functional catalyst precursor was denoted as Cat5.

### APPLICATION EXAMPLE 2

The performance of the catalyst precursors prepared in Examples 4-5 were tested according to the method in Application Example 1, and the test results are shown in Table 2.

TABLE 2
Test results of the performance of the catalyst precursors prepared in Examples 4-5 after activation

| Catalyst | Mass selectivity % | | CO |
|---|---|---|---|
| precursor | Methyl acetate | Methyl acetate | conversion % |
| Cat2 | 10.3 | 88.7 | 11.4 |
| Cat4 | 32.2 | 66.2 | 7.2 |
| Cat5 | 59.9 | 39.2 | 6.9 |

It can be seen from Table 2 that the dual-functional catalyst prepared by using 3-aminopropyltriethoxysilane as the silane coupling agent has better catalytic performance than that prepared by using 3-aminopropyltrimethoxysilane or vinyltrichlorosilane as the silane coupling agent.

### EXAMPLE 6

A dual-functional catalyst precursor was prepared according to the method of Example 4, except that the HNO₃ solution with a concentration of 5 wt % was replaced with a sulfuric acid solution with a concentration of 5 wt %. The final obtained dual-functional catalyst precursor was denoted as Cat6.

### EXAMPLE 7

A dual-functional catalyst precursor was prepared according to the method of Example 4, except that the HNO₃ solution with a concentration of 5 wt % was replaced with an HCl solution with a concentration of 5 wt %. The final obtained dual-functional catalyst precursor was denoted as Cat7.

### EXAMPLE 8

A dual-functional catalyst precursor was prepared according to the method of Example 4, except that the HNO₃ solution with a concentration of 5 wt % was replaced with an NaOH solution with a concentration of 5 wt %. The final obtained dual-functional catalyst precursor was denoted as Cat8.

### COMPARATIVE EXAMPLE 6

A dual-functional catalyst precursor was prepared according to the method of Example 4, except that the step of immersing the metal oxide in an HNO₃ solution with a concentration of 5 wt % for 2 h was omitted, i.e., the metal oxide was directly placed in the silane coupling agent mixed solution for the subsequent treatment. The final dual-functional catalyst precursor obtained was denoted as Cat6#.

### APPLICATION EXAMPLE 3

The performance of the catalyst precursor prepared in Examples 6-8 and Comparative Example 6 was tested according to the method of Application Example 1, except that the composition of the reaction raw material synthesis gas is as follows: a volume fraction of dimethyl ether is 10%, a volume fraction of CO is 85%, and a volume fraction of H₂O is 5%. The test results of the performance are shown in Table 3.

TABLE 3
Test results of performance of the catalyst precursors prepared in Examples 6-7 and Comparative Example 6 after activation

| Catalyst | Mass selectivity % | | CO |
|---|---|---|---|
| precursor | Methyl acetate | Methyl acetate | conversion % |
| Cat6 | 18.1 | 79.4 | 10.6 |
| Cat6^{#} | 92.0 | 6.4 | 1.8 |
| Cat7 | 37.6 | 58.9 | 7.7 |
| Cat8 | 32.9 | 62.7 | 7.5 |

It can be seen from Table 3 that the finally obtained catalyst using a nitric acid solution with a concentration of 5 wt % to modify the metal composite oxide has a greatly better performance than that of the catalyst prepared from the unmodified metal oxide; and the modification effect of the nitric acid solution with a concentration of 5 wt % is better than that of the HCl solution with a concentration of 5 wt %.

The above are only the preferred embodiments of the present disclosure. It should be pointed out that for those of ordinary skill in the art, without departing from the principle of the present disclosure, several improvements and modifications could be made, and these improvements and modifications should also fall within the protection scope of the present disclosure.

1. A carbonylation-dehydration dual-functional catalyst precursor, comprising
a modified silica-aluminum molecular sieve having an 8-member ring channel structure; and
a modified metal oxide loaded on the modified silica-aluminum molecular sieve having an 8-member ring channel structure by coupling, the coupling being performed using a silane coupling agent, wherein
a modified component in the modified silica-aluminum molecular sieve having an 8-member ring channel structure comprises at least one selected from the group consisting of copper oxide, zinc oxide and iron oxide, and has a loading amount of 0.5-5 wt %, based on a metal mass of the modified component; and
the modified metal oxide is prepared by modifying a composite metal oxide with an acid solution or an alkali solution, wherein the composite metal oxide is prepared based on a co-precipitation-calcination method, and the composite metal oxide is at least one selected from the group consisting of CuₐZn₁₋ₐO_{y}, Cu_{b}Mn_{1-b}O_{y} and CuₘZnₙAl₁₋ₘ₋ₙO_{y}, wherein 0<a<1, 0<b<1, 0<m<1, 0<n<1, 0<m+n<1, and y=1.0-2.0.

2. The carbonylation-dehydration dual-functional catalyst precursor of claim 1, wherein a molecular sieve in the modified silica-aluminum molecular sieve having an 8-member ring channel structure is an MOR molecular sieve or an FER molecular sieve.

3. The carbonylation-dehydration dual-functional catalyst precursor of claim 1, wherein a mass ratio of the modified silica-aluminum molecular sieve having an 8-member ring channel structure to the modified metal oxide is in a range of 1:(0.05-0.5).

4. The carbonylation-dehydration dual-functional catalyst precursor of claim 1, wherein the silane coupling agent comprises at least one selected from the group consisting of 3-aminopropyltrimethoxysilane, 3-aminopropyltriethoxysilane, vinyltrichlorosilane, vinyl tris (2-methoxyethoxy) silane, and vinyltriacetoxysilane.

5. The carbonylation-dehydration dual-functional catalyst precursor of claim 1, wherein the acid solution comprises at least one selected from the group consisting of a hydrochloric acid solution, a nitric acid solution, a sulfuric acid solution, an acetic acid solution, and a citric acid solution, and the acid solution has a concentration of 0.1-6 wt %; and
the alkali solution comprises at least one selected from the group consisting of a sodium hydroxide solution, a potassium hydroxide solution, a calcium hydroxide solution or an ammonia water, and the alkali solution has a concentration of 0.1-8 wt %.

6. A method for preparing the carbonylation-dehydration dual-functional catalyst precursor of claim 1, comprising the following steps:
pretreating the modified metal oxide with the silane coupling agent to obtain a pretreated modified metal oxide; and
coupling the pretreated modified metal oxide and the modified silica-aluminum molecular sieve having an 8-member ring channel structure to obtain the carbonylation-dehydration dual-functional catalyst precursor.

7. The method of claim 6, wherein the silane coupling agent is used in a form of a silane coupling agent solution, and the silane coupling agent solution has a concentration of 0.5-50 wt %; a solvent of the silane coupling agent solution is an alcohol-water mixed solvent, and a volume ratio of the alcohol to water in the alcohol-water mixed solvent is in a range of (1-200):10.

8. The method of claim 7, wherein a ratio of the volume of the silane coupling agent solution to the total mass of the modified metal oxide and the modified silica-aluminum molecular sieve having an 8-member ring channel structure is in a range of (1-20)mL:1 g.

9. A carbonylation-dehydration dual-functional catalyst, which is prepared by activating the carbonylation-dehydration dual-functional catalyst precursor of claim 1 in an H₂—N₂ mixed gas.

10. A method for production of methyl acetate from synthesis gas through dimethyl ether by using the carbonylation-dehydration dual-function catalyst of claim 9, comprising in the presence of the carbonylation-dehydration dual-functional catalyst, using synthesis gas as a raw material to produce methyl acetate, wherein a reaction for producing methyl acetate through dimethyl ether is performed at a temperature of 170-280° C.

11. The carbonylation-dehydration dual-functional catalyst precursor of claim 2, wherein a mass ratio of the modified silica-aluminum molecular sieve having an 8-member ring channel structure to the modified metal oxide is in a range of 1:(0.05-0.5).

12. The method of claim 6, wherein a molecular sieve in the modified silica-aluminum molecular sieve having an 8-member ring channel structure is an MOR molecular sieve or an FER molecular sieve.

13. The method of claim 6, wherein a mass ratio of the modified silica-aluminum molecular sieve having an 8-member ring channel structure to the modified metal oxide is in a range of 1:(0.05-0.5).

14. The method of claim 6, wherein the silane coupling agent comprises at least one selected from the group consisting of 3-aminopropyltrimethoxy silane, 3-aminopropyltriethoxysilane, vinyltrichlorosilane, vinyl tris (2-methoxyethoxy) silane, and vinyltriacetoxysilane.

15. The method of claim 6, wherein the acid solution comprises at least one selected from the group consisting of a hydrochloric acid solution, a nitric acid solution, a sulfuric acid solution, an acetic acid solution, and a citric acid solution, and the acid solution has a concentration of 0.1-6 wt %; and
the alkali solution comprises at least one selected from the group consisting of a sodium hydroxide solution, a potassium hydroxide solution, a calcium hydroxide solution or an ammonia water, and the alkali solution has a concentration of 0.1-8 wt %.

16. The carbonylation-dehydration dual-functional catalyst of claim 9, wherein a molecular sieve in the modified silica-aluminum molecular sieve having an 8-member ring channel structure is an MOR molecular sieve or an FER molecular sieve.

17. The carbonylation-dehydration dual-functional catalyst of claim 9, wherein a mass ratio of the modified silica-aluminum molecular sieve having an 8-member ring channel structure to the modified metal oxide is in a range of 1:(0.05-0.5).

18. The carbonylation-dehydration dual-functional catalyst of claim 9, wherein the silane coupling agent comprises at least one selected from the group consisting of 3-aminopropyltrimethoxysilane, 3-aminopropyltriethoxysilane, vinyltrichlorosilane, vinyl tris (2-methoxyethoxy) silane, and vinyltriacetoxysilane.

19. The carbonylation-dehydration dual-functional catalyst of claim 9, wherein the acid solution comprises at least one selected from the group consisting of a hydrochloric acid solution, a nitric acid solution, a sulfuric acid solution, an acetic acid solution, and a citric acid solution, and the acid solution has a concentration of 0.1-6 wt %; and
the alkali solution comprises at least one selected from the group consisting of a sodium hydroxide solution, a potassium hydroxide solution, a calcium hydroxide solution or an ammonia water, and the alkali solution has a concentration of 0.1-8 wt %.

20. The carbonylation-dehydration dual-functional catalyst of claim 9, wherein the carbonylation-dehydration dual-functional catalyst precursor is prepared by a method comprising the following steps:
pretreating the modified metal oxide with the silane coupling agent to obtain a pretreated modified metal oxide; and
coupling the pretreated modified metal oxide and the modified silica-aluminum molecular sieve having an 8-member ring channel structure to obtain the carbonylation-dehydration dual-functional catalyst precursor.
